(19) United States
(12) Patent Application Publication
(10) Pub. No.: US 2026/0027185 A1
(43) Pub. Date: Jan. 29, 2026

(54) COMPOSITION FOR PREVENTING OR TREATING HERNIATED INTERVERTEBRAL DISC OR PAIN CAUSED THEREBY, COMPRISING HYDROGEL PATCH

(71) Applicant: SUPINE THERAPEUTICS CO., LTD., Ulsan (KR)

(72) Inventors: Ji Yeon SEO, Gimhae-si (KR); Jeong Beom KIM, Ulsan (KR)

(73) Assignee: SUPINE THERAPEUTICS CO., LTD., Ulsan (KR)

(21) Appl. No.: 18/833,324

(22) PCT Filed: Oct. 26, 2021
(86) PCT No.: PCT/KR2021/015118

§ 371 Date: Apr. 9, 2025

(30) Foreign Application Priority Data
Oct. 26, 2020 (KR) 10-2020-0139534

(51) Int. Cl. A61K 38/36; A61K 9/70; A61K 31/728; A61K 38/18; A61K 38/39; A61K 38/48; A61P 29/00

(52) U.S. Cl. CPC A61K 38/363; A61K 9/7023; A61K 31/728; A61K 38/1825; A61K 38/185; A61K 38/1866; A61K 38/1875; A61K 38/39; A61K 38/4833; A61P 29/00

(57) ABSTRACT

Provided is a composition for the treatment of herniated intervertebral disc or pain resulting therefrom, including a hydrogel patch, which shows the effects of improving neurological grade before and after the procedure, improving behavioral ability before and after the procedure, and recovering sensory ability, in a disc animal model. Accordingly, the composition can be useful for the prevention or treatment of the herniated intervertebral disc or pain resulting therefrom in a minimally invasive method.

### TECHNICAL FIELD

The present disclosure relates to a composition for treating herniated intervertebral disc or pain resulting therefrom, including a hydrogel patch.

### BACKGROUND ART

An intervertebral disc is an intervertebral tissue that exists between the vertebrae of the spine in vertebrates, and is also called a spinal disc or intervertebral disc. Due to the structure, the intervertebral disc has strong elasticity and expandability, and plays an important role in the flexion and extension movement of the spine and the support of the body based on the spine. Each intervertebral disc is thickest in the middle thereof. The outer part of the disc contains the annulus fibrosus, which consists of connective tissue and fibrous cartilage, and the inner center thereof contains the nucleus pulposus, which consists of a group of chondrocytes and soft fibrous cartilage and contains a large amount of water.

Meanwhile, as people age, intervertebral discs inevitably undergo degenerative changes, and these processes can begin to appear in the late teens or early 20s. As the intervertebral disc degenerates, a fissure occurs in the annulus fibrosus surrounding the edge of the intervertebral disc, and the nucleus pulposus within the intervertebral disc may prolapse beyond the border of the vertebrae. The disease in which the intervertebral disc protrudes and causes back pain and nerve symptoms, is called herniated intervertebral disc, spinal disc disease, spinal disc, or lumbar disc.

The most prominent symptoms of patients with lumbar herniated intervertebral disc are lower back pain and radiating pain, which is aching and tingling in the feet. The herniated intervertebral disc irritates the nerve root, causing sensory abnormalities in the feet where the nerve root is distributed. When the 5th lumbar nerve root is irritated, paresthesia may occur on the inside of the instep, and when the 1st lumbar nerve root is irritated, paresthesia may occur on the outside of the instep. In most cases, decreased sensation or numbness may occur. In some cases, hyperalgesia may occur. Muscle strength also weakens, and in the case of fifth nerve root disease, the foot extensors become weak and walking on heels becomes difficult. In rare cases, if the protruding nucleus pulposus is large and centrally located, urinary or sexual dysfunction and paralysis of the lower extremities may occur.

Treatment of herniated intervertebral disc can be divided into conservative methods and surgical methods. When choosing a treatment method, several conditions such as the duration of symptoms, intensity of pain, number of recurrences, patient's occupation, age, and gender, and workload need to be considered. Surgical therapy is recommended when there is unbearable pain that is not effective even with conservative treatment for 6 to 12 weeks, when paralysis of the lower extremities does not improve or progresses, when bowel and urination problems occur, or when pain frequently recurs and interferes with daily life and with the use of leisure time.

However, since there are many cases of recurrence even after surgical treatment, there is a need to develop a therapeutic agent for the treatment of herniated intervertebral discs that is minimally invasive and has lasting effects.

Accordingly, the inventors of the present application found that when a hydrogel patch containing fibrinogen, laminin, and hyaluronic acid was applied to a canine disc model, the effects of improving neurological grade, improving behavioral ability, and recovering sensory ability occurred, and thereby completed the present disclosure.

### DETAILED DESCRIPTION OF THE DISCLOSURE

### Technical Problem

The objective of the present disclosure is to provide a pharmaceutical composition for the treatment of herniated intervertebral disc or pain resulting therefrom, which has a lasting effect through a minimally invasive method.

Another objective is to provide a pharmaceutical composition that is effective in preventing or treating herniated intervertebral disc or pain resulting therefrom, including hydrogel or hydrogel patch.

### Technical Solution to Problem

An aspect provides a pharmaceutical composition for preventing or treating herniated intervertebral disc or pain resulting therefrom including, as an active ingredient, fibrin and/or fibrinogen; laminin or laminin-derived peptides or proteins; and/or a hyaluronic acid or a salt thereof.

Another aspect provides a pharmaceutical composition for preventing or treating herniated intervertebral disc or pain resulting therefrom including, as an active ingredient, hydrogel or hydrogel patch including fibrin and/or fibrinogen; laminin or laminin-derived peptides or proteins; and/or a hyaluronic acid or a salt thereof.

Another aspect provides a method of treating herniated intervertebral disc or pain resulting therefrom, including administering the hydrogel, the hydrogel patch, or the pharmaceutical composition to a subject in a pharmaceutically effective amount.

Another aspect provides use of the hydrogel, the hydrogel patch, or the pharmaceutical composition for use in the preparation of a therapeutic agent for the treatment of herniated intervertebral disc or pain resulting therefrom.

The hydrogel, the hydrogel patch, or the pharmaceutical composition may include: fibrin and/or fibrinogen; laminin or laminin-derived peptides or proteins; and/or a hyaluronic acid or a salt thereof. In some embodiments, the hydrogel, the hydrogel patch, or the pharmaceutical composition may include: fibrin or fibrinogen; laminin or laminin-derived peptides or proteins; and a hyaluronic acid or a salt thereof.

In an embodiment, when a hydrogel patch containing fibrinogen, laminin, and hyaluronic acid was applied to the compressed spinal cord of a canine disc animal model, the effects of improving neurological grade, improving behavioral ability, and recovering sensory ability occurred. Therefore, the hydrogel patch may be used as a composition for preventing or treating herniated intervertebral disc or pain resulting therefrom.

The herniated intervertebral disc may include, but is not limited to, cervical or lumbar herniated intervertebral disc. The pain caused by the herniated intervertebral disc may include, but is not limited to, one or more selected from the group consisting of neck pain, chronic neck pain, lower back pain, chronic lower back pain, radiating pain, and coccydynia. Specifically, the pain caused by the herniated intervertebral disc may include, in addition to pain occurs in a specific area from the waist to the feet, radiating pain that radiates from below the knees to the tips of the toes, and lower extremity pain that occurs when a herniated intervertebral disc directly stimulates the nerve root.

The term “pharmaceutically effective amount” refers to an amount of a composition effective in preventing or treating herniated intervertebral disc or pain resulting therefrom, which is sufficient to treat the disease at a reasonable benefit/risk ratio applicable to medical treatment and does not cause side effects. The level of the effective amount is determined by factors including the patient's health status, type and severity of the disease, activity of the drug, sensitivity to the drug, administration method, administration time, administration route and excretion rate, treatment period, drugs to be combined or concurrently used, and other factors well known in the medical field.

The terms “administering,” “applying,” “introducing,” and “implanting” are used interchangeably and refer to placement of a patch or composition according to an embodiment into a subject by a method or route that results in at least partial localization of the patch or composition according to an embodiment to the desired site.

The term “patch” may refer to a member that has a certain shape and can be applied, attached, or brought into contact with a target area.

In an embodiment, the hydrogel patch or composition may have properties between solid and liquid. The hydrogel patch or composition may be amorphous, spherical, hemispherical, disc-shaped, or cylindrical. In some embodiments, for example, the diameter of the hydrogel patch may be 0.05 mm to 10 cm, 0.1 mm to 5 cm, 0.1 mm to 3 cm, or 0.2 mm to 1.5 cm, and may be provided in such sizes or shapes. In addition, the hydrogel patch may have a shape which is to be changed corresponding to the shape of an area on which the hydrogel patch is to be applied by applying, implanting, attaching, or contacting on the target site (for example, a compressed spinal cord area).

In other embodiments, the hydrogel patch or composition may be solid (including powder), semi-solid, or liquid. Additionally, for example, the hydrogel patch or composition may undergo a reversible phase transition into a solid (including powder), semi-solid, or liquid state (for example, depending on temperature change). Since the hydrogel patch may undergo a reversible phase transition depending on surrounding circumstances such as temperature conditions, the hydrogel patch according to an embodiment may be produced and provided in a solid (including powder) or liquid state, and before administration to the target area, it can be converted into a hydrogel patch and used during or after administration. For example, the hydrogel patch is provided in a sol state containing fibrinogen, laminin, and hyaluronic acid, so that a user can manufacture hydrogel patch according to an embodiment before use by using a material (for example, thrombin) that can convert fibrinogen into fibrin. Therefore, the hydrogel patch according to an embodiment may be provided in the form of a composition including fibrin and/or fibrinogen; laminin; and/or compositions comprising hyaluronic acid, for example, in the form of a prodrug in a solid (powder), liquid (sol), or semi-solid composition. A composition provided in the form of a prodrug may be prepared or modified into a hydrogel patch and act in vivo. Additionally, the hydrogel patch according to an embodiment may further include thrombin, or thrombin may be provided together as a kit.

In some embodiments, the hydrogel patch or composition may be porous. In detail, the surface of the hydrogel patch may have porosity (micropores). Without being limited to a specific theory, the hydrogel patch according to an embodiment has porosity, which may lead to the increase in the interaction between active materials.

In an embodiment, the hydrogel, the hydrogel patch, or the pharmaceutical composition may include fibrin and/or fibrinogen. The final pharmacological substance that acts in vivo includes fibrin, but instead of fibrin, fibrinogen may be used in the form of a prodrug. Additionally, depending on the amount of the substance that converts fibrinogen into fibrin, a portion of the hydrogel patch or composition according to an embodiment may include fibrinogen or thrombin. Accordingly, another aspect includes a prodrug including: fibrinogen; laminin; and/or a hyaluronic acid. The fibrin or fibrinogen may be included at a concentration of 0.1 mg/ml to 50 mg/ml, 0.1 mg/ml to 25 mg/ml, 0.5 mg/ml to 25 mg/ml, 1 mg/ml to 25 mg/ml, 1 mg/ml to 20 mg/ml, 5 mg/ml to 20 mg/ml, 0.5 mg/ml to 15 mg/ml, 1 mg/ml to 15 mg/ml, 3 mg/ml to 15 mg/ml, 5 mg/ml to 15 mg/ml, 7 mg/ml to 12 mg/mi, or 8 mg/ml to 12 mg/ml.

Fibrinogen glycoprotein is a hexamer consisting of soluble α, β, and γ subunits produced by hepatocytes. Fibrinogen reacts with thrombin enzyme and undergoes a phase transition from soluble to insoluble fibrin polymer fiber.

The thrombin enzyme is a serine protease and is an enzyme that transforms soluble fibrinogen into insoluble fibrin. The thrombin may change fibrinogen into fibrin, from the sol-state hydrogel to gel-state hydrogel.

The term “laminin” is an extracellular matrix protein constituting the basal lamina and may refer to a heterotrimeric protein consisting of α, β, and γ subunits. In some embodiments, laminin may include not only full-length laminin protein but also laminin-derived peptides or proteins. For example, the laminin may be Iaminin-1, Iaminin-2, Iaminin-3, Iaminin-4, laminin-5A, Iaminin-5B, Iaminin-6, Iaminin-7, Iaminin-8, Iaminin-9, and Iaminin-10, Iaminin-11, Iaminin-12, Iaminin-14, or Iaminin-15. In some embodiments, the laminin-derived peptide may be an α chain, γ chain, or β chain. The laminin may be included at a concentration of 1 μg/ml to 100 μg/ml, 2 μg/ml to 80 μg/ml, 5 μg/ml to 50 μg/ml, 10 μg/ml to 70 μg/ml, 20 μg/ml to 50 μg/ml, or 20 μg/ml to 40 μg/ml.

The hyaluronic acid is a type of glycosaminoglycan called hyaluronan, and a polysaccharide of disaccharide bonds consisting of glycosidic bonds in which D-glucuronic acid and N-acetyl-D-glucosamine undergo the changes of β-(1→4) and β-(1→3), and has various molecular weights depending on the length of the disaccharide bond. In an embodiment, the molecular weight of hyaluronic acid may be 5,000 Da to 20,000,000 Da. In some embodiments, the molecular weight of hyaluronic acid may be 0.5×10⁶ Da to 4.0×10⁶ Da, 1.0×10⁶ Da to 2.0×10⁶ Da, or 1.5×10⁶ Da to 1.8×10⁶ Da. The hyaluronic acid may be included at a concentration of 10 μg/ml to 10 mg/ml, 10 μg/ml to 5 mg/ml, 100 μg/ml to 3 mg/ml, 500 μg/ml to 5 mg/ml, 500 μg/ml to 8 mg/ml, 500 μg/ml to 4 mg/ml, 500 μg/ml to 3 mg/ml, 300 μg/ml to 3 mg/ml, 1 mg/ml to 8 mg/ml, 1 mg/ml to 5 mg/ml, or 1 mg/ml to 3 mg/ml.

In some embodiments, the hydrogel, or the hydrogel patch or composition may further include cell growth factors. The cell growth factors may include a nerve cell growth factor, a vascular endothelial cell growth factor, a fibroblast growth factor, a bone morphogenetic protein, an epidermal growth factor, a hepatocyte growth factor, a transforming growth factor, or a combination thereof. In some embodiments, the cell growth factors may include placental growth factor, macrophage colony-stimulating factor, granulocyte-macrophage colony-stimulating factor, neuropilin, fibroblast growth factor (FGF)-1, FGF-2 (bFGF), FGF-3, FGF-4, FGF-5, FGF-6, erythropoietin, BMP-2, BMP-4, BMP-7, TGF-beta, IGF-1, osteopontin, pleiotropin, activin, endocellin-1, or a combination thereof. The nerve cell growth factors may include at least one selected from the group consisting of a brain-derived neurotropic factor (BDNF), a glial cell derived neurotropic factor (GDNF), a ciliary neurotropic factor (CNTF), a basic fibroblast growth factor (bFGF), a cyclic adenocyne monophosphate (cAMP), neurotropin (NT), neurotropin-3 (NT3), neurotropin-4 (NT4), triiodo-L-thyronine (T3), sonic hedgehog (SHH), and a platelet-derived growth factor (PDGF). The vascular endothelial growth factor may include vascular endothelial growth factor (VEGF)-A, VEGF-B, VEGF-C, VEGF-D, or VEGF-E.

The concentration of the cell growth factor included in the hydrogel, hydrogel patch, or the composition may vary depending on the type of growth factor, and may be 1 ng/ml to 1,000 ng/ml or 0.1 μM to 100 μM. The cell growth factor may increase the effects of a hydrogel patch or composition containing fibrin, laminin, and hyaluronic acid of protection or regeneration of spinal nerves which are damaged by being compressed by a disc.

In other embodiments, the hydrogel or the hydrogel patch or composition may include collagen or may not substantially include collagen. Without being limited to a particular theory, the collagen may or may not be substantially included as a component of the hydrogel or the hydrogel patch or composition, but in some aspects, the latter case may be more advantageous than the former case.

Additionally, the hydrogel, or the hydrogel patch or composition may not substantially include cells.

The wording “not substantially include” refers to the feature in which collagen or cells are included to the extent that they do not affect the activity, or pharmacological activity of the hydrogel, or the hydrogel patch or composition, or are not included at all. The hydrogel patch according to an embodiment does not substantially include cells, and thus may be distinguished from cell therapy products generally used for regeneration of damaged tissues.

In an embodiment, the hydrogel or the hydrogel patch or composition may include: fibrin and/or fibrinogen; laminin or laminin-derived peptide; and a hyaluronic acid or a salt thereof. In addition, one or more of the ingredients described above may be additionally included. For example, the hydrogel, or the hydrogel patch or composition may include fibrin and/or fibrinogen; laminin or laminin-derived peptide; a hyaluronic acid or a salt thereof; optionally thrombin; optionally collagen; and optionally, cell growth factors.

The dosage of the composition according to an embodiment may be 0.01 mg to 10,000 mg, 0.1 mg to 1000 mg, 1 mg to 100 mg, 0.01 mg to 1000 mg, 0.01 mg to 100 mg, 0.01 mg to 10 mg, or 0.01 mg to 1 mg. However, the dosage may be prescribed in various ways depending on factors such as a formulation method, an administration method, patient's age, weight, gender, pathological condition, food, administration time, an administration route, an excretion rate, and reaction sensitivity, and those skilled in the art may take these factors into consideration to adjust the dosage appropriately. The number of administrations may be one time, or two or more times within the range of clinically acceptable side effects, and the administration site may be administered at one or two or more sites. For animals other than humans, the dosage per kg may be the same as that for humans, or the dosage may be converted using, for example, the volume ratio (for example, average value) of an organ (the heart, etc.) of the target animal to that of human and the adjusted dosage is administered thereto. Possible routes of administration include oral, sublingual, parenteral (for example, subcutaneous, intramuscular, intraarterial, intraperitoneal, intrathecal, or intravenous), rectal, topical (including transdermal), inhalation, and injection routes, or insertion of implantable device or a substance. Examples of animals subject to treatment according to an embodiment include humans and other mammals, specifically humans, monkeys, mice, rats, rabbits, sheep, cows, dogs, horses, pigs, etc.

The pharmaceutical composition according to an embodiment may include a pharmaceutically acceptable carrier and/or additive. For example, the pharmaceutical composition may include sterile water, physiological saline, common buffers (phosphoric acid, citric acid, other organic acids, etc.), stabilizers, salts, antioxidants (ascorbic acid, etc.), surfactants, suspending agents, isotonic agents, or preservatives. For topical administration, the pharmaceutical composition may also include combinations with organic materials such as biopolymers and inorganic materials such as hydroxyapatite, particularly, collagen matrices, polylactic acid polymers or copolymers, polyethylene glycol polymers or copolymers, and chemical derivatives thereof.

According to an embodiment, depending on the administration method or formulation, the pharmaceutical composition may appropriately include a suspending agent, a solubilizing agent, a stabilizer, an isotonic agent, a preservative, an anti-adsorption agent, a surfactant, diluent, an excipient, a pH adjuster, an analgesic agent, a buffer, a reducing agent, an antioxidant, etc. Pharmaceutically acceptable carriers and agents suitable for the present disclosure, including those provided as an example, are described in detail in this document [Remington's Pharmaceutical Sciences, 19th ed., 1995]. The pharmaceutical composition according to an embodiment may be formulated in unit dosage form or by placing the same in a multi-capacity container by using a pharmaceutically acceptable carrier and/or excipient according to a method that can be easily performed by those skilled in the art to which the present disclosure pertains. The formulation may be in the form of a solution, suspension or emulsion in an oil or aqueous medium, or in the form of powder, granules, tablets or capsules.

The composition may be formulated as an oral or parenteral dosage form. Oral dosage form may be granules, powder, solutions, tablets, capsules, dry syrup, or combinations thereof. The parenteral dosage form may be an injection or an external dermatological agent for skin.

External dermatological agents may be creams, gels, ointments, skin emulsifiers, skin suspensions, transdermal patches, drug-containing bandages, lotions, or combinations thereof. The external dermatological agents may be appropriately mixed, according to need, with ingredients commonly used in external dermatological agents, for example, cosmetics, pharmaceuticals, and quasi-drugs. Examples of such ingredients are water-based ingredients, oil-based ingredients, powder ingredients, alcohols, moisturizers, thickeners, ultraviolet absorbers, whitening agents, preservatives, antioxidants, and surfactants, fragrance, colorants, various skin nutrients, or a combination thereof. For the external dermatological agents, metal sequestrants such as disodium edetate, trisodium edetate, sodium citrate, sodium polyphosphate, sodium metaphosphate, and gluconic acid; hot water extract of fruit, such as caffeine, tannin, belafamil, licorice extract, glablidin, and calin; drugs such as various herbal medicines, tocopherol acetate, glytylitinic acid, and tranexamic acid and derivatives or salts thereof; or saccharides such as vitamin C, magnesium ascorbate phosphate, ascorbate glucoside, arbutin, kojic acid, glucose, fructose, and trehalose, may also be appropriately mixed.

Additionally, the composition may be a quasi-drug composition. The term “quasi-drug” refers to an article corresponding to one of: fibers, rubber products or products similar thereto, used for the purpose of treating, alleviating, treating or preventing diseases of humans or animals; one that has a weak effect on the human body or does not directly act on the human body, and is not instruments or machines and products similar thereto; and preparations used for sterilization, insecticide, and purposes similar thereto to prevent infection, and an article excluding: articles that are used for the purpose of diagnosing, treating, alleviating, treating, or preventing diseases of humans or animals, and are not instruments, machines, or apparatuses; and articles that are used for the purpose of having a pharmacological effect on the structure and function of humans or animals, and are not instruments, machines, or apparatuses, and may also include external dermatological agents for the skin and personal hygiene products. When the hydrogel, or the hydrogel patch or composition is added to a quasi-drug composition for the purpose of preventing or alleviating herniated intervertebral disc or pain resulting therefrom, the hydrogel, or the hydrogel patch or composition may be added as is or used together with other quasi-drug ingredient, and can be used appropriately according to conventional methods. The mixing amount of the active ingredient may be appropriately determined depending on the purpose of use (prevention, health, or therapeutic treatment).

Another aspect provides a method of preparing a hydrogel patch including adding thrombin to a composition in a sol state, the composition including fibrinogen; laminin or laminin-derived peptides; and a hyaluronic acid or a pharmaceutically acceptable salt thereof.

The method may further include mixing a cell growth factor with the sol composition.

The method may further include performing secondary gelation by adding thrombin after gelling by adding thrombin. The gelation may be performed at 10° C. to 40° C. for 5 minutes to 3 hours. The hydrogel patch may be manufactured in various shapes or sizes depending on the shape of a frame.

The method may further include cold-preserving or cryopreserving the hydrogel patch in a solution at 4° C. to −210° C. The solution may include dimethyl sulfoxide (DMSO), but any solution may be used herein as long as it does not substantially change the chemical or physical properties of the hydrogel patch. The hydrogel patch does not substantially change the form or activity thereof even when stored at low temperature or cryopreserved.

Another aspect provides a method of cold-preserving or cryopreserving the hydrogel patch at 4° C. to −210° C. in a solution (for example, DMSO).

The hydrogel patch, fibrin and/or fibrinogen, laminin, hyaluronic acid or the cell growth factor are as described above.

Redundant descriptions are omitted in consideration of the complexity of the present specification, and terms not otherwise defined in this specification have meanings commonly used in the technical field to which the present disclosure pertains.

### Advantageous Effects of Disclosure

According to the hydrogel patch or composition according to an aspect, the effects of improving neurological grades before and after the procedure and improving behavioral ability before and after the procedure occur in a disc animal model. Thus, the hydrogel patch or composition can be useful for the treatment of herniated intervertebral disc or pain resulting therefrom.

In addition, the hydrogel patch or composition for disc treatment according to one aspect promotes recovery of paralyzed motor and sensory abilities of disc patients, and thus has a therapeutic effect for herniated intervertebral disc, which is difficult to treat with surgery alone.

### BRIEF DESCRIPTION OF DRAWINGS

FIG. 1 shows an image of a hydrogel patch for disc treatment manufactured according to one aspect.

FIG. 2 shows check-up test results of Case 1, a canine disc model: the paw replacement test (left), back pain (middle), and ambulatory test (right).

FIG. 3 shows the results of MRI of canine disc model Case 2 according to one aspect to confirm the area and severity of disc occurrence.

FIG. 4 shows check-up test results of case 2, a canine disc model: the paw replacement test (left), extensor postural thrust (middle), and ambulatory test (right).

FIG. 5 shows an image of a hydrogel patch for disc treatment manufactured according to one aspect applied to the compressed spinal cord area of a canine disc model.

FIG. 6 shows check-up results after applying a hydrogel patch for disc treatment to canine disc model case 1 according to one aspect: paw replacement test (left), back pain (middle), and ambulatory test (right).

FIG. 7 shows check-up results after applying a hydrogel patch for disc treatment to canine disc model case 2 according to one aspect: paw replacement test (left), extensor postural thrust (middle), and ambulatory test (right).

### MODE OF DISCLOSURE

Hereinafter, the present disclosure will be explained in more detail by examples. However, these examples are for illustrative purposes only and the scope of the present disclosure is not limited by these examples.

### Example 1. Manufacturing of Hydrogel Patches

### Example 1.1 Hydrogel Formulation

20 mg/ml of fibrinogen (Sigma, F8630), 5 mg/ml of hyaluronic acid (Sigma, 53747), and 200 U/ml of thrombin were each dissolved in a sol state in PBS. Afterwards, sol-state fibrinogen, laminin (Thermofisher, 23017-015), and hyaluronic acid were mixed at concentrations of 10 mg/ml, 30 μg/ml, and 1.5 mg/ml, respectively, to produce a sol-state hydrogel.

### Example 1.2 Preparation of Hydrogel Patch

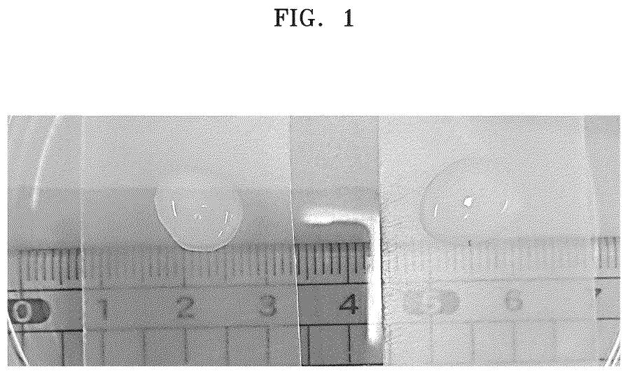

A circular frame having the diameter of 0.3 mm to 0.8 mm was prepared using sterilized parafilm, 13.3 U/ml thrombin (Sigma, T4648) in sol was spread, and then 200 μl of the sol hydrogel mixed in Example 1.1 was added to the round frame, and mixed with thrombin. Afterwards, a hydrogel patch for disc treatment was produced through sol-gel phase transition of cross-linker reaction gelation at 37° C. for 1 hour (FIG. 1).

### Experimental Example 1. Confirmation of the Disc Treatment Effect of the Hydrogel Patch

In order to confirm the disc treatment effect of the hydrogel patch prepared in Example 1, the following four cases of canine disc models were used.

TABLE 1

| | | Surgical site | Neurological grade |
|---|---|---|---|
| Cases | Breed | (Degree of compression) | (NG) |
| 1 | Dachshund | T12-13 Type I | 5 |
| | | (Severe) | |
| 2 | French Bulldog | L3-4, L4-5 Type I | 4-5 |
| | | (Severe) | |
| | | L1-2, L2-3 Type II | |
| | | (Moderate) | |
| 3 | Dachshund | T13-L1 Type I | 4 |
| | | (Severe) | |
| 4 | Dachshund | L1-2 Type I | 4 |
| | | (Severe) | |

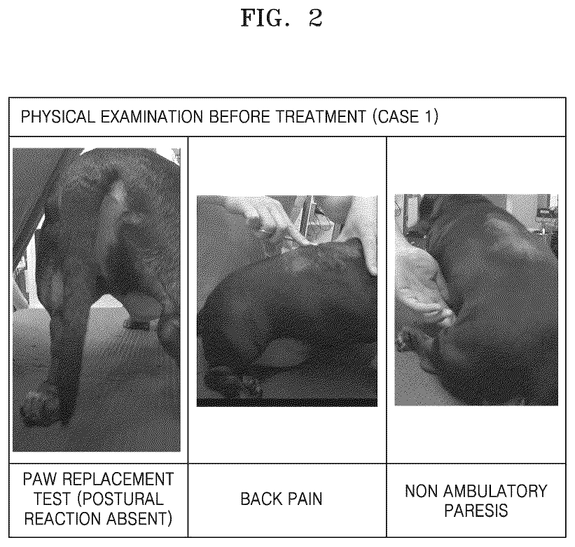

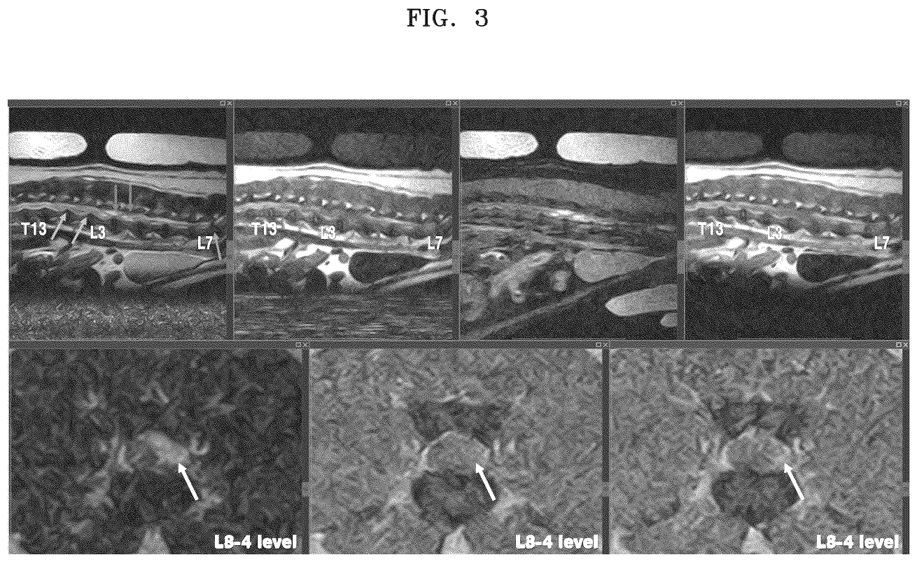

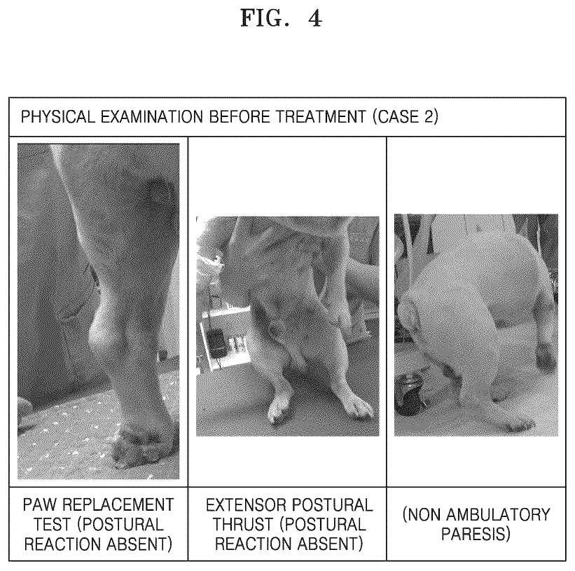

As a result of performing the check-up of the Dachshund disc model in Case 1, as shown in FIG. 2, i) in the paw replacement test, no postural response was observed in both feet, ii) back pain occurred, and iii) in the ambulatory test, walking on both feet was impossible, that is, symptoms of paralysis occurred. In Case 2, the French Bulldog disc model was examined by MRI to confirm the location and severity of the disc, and as shown in FIG. 3, there was severe spinal cord compression from the Type I disc in lumbar 3-5 and there was moderate spinal cord compression from the Type I disc in lumbar 1-3. In addition, as a result of performing the check up for the French Bulldog disc model in Case 2, as shown in FIG. 4, i) in the paw replacement test, the left foot did not show postural response, ii) in the extensor postural thrust test for evaluating: whether the foot is straightened and at the same time touches the ground until the foot descends and touches the ground; and the movement, speed, and distance of the hind foot to naturally go back after the foot touched the ground, the hind foot did not show postural response, and iii) in the ambulatory test, walking on the left foot was impossible, that is, the left foot showed symptoms of paralysis.

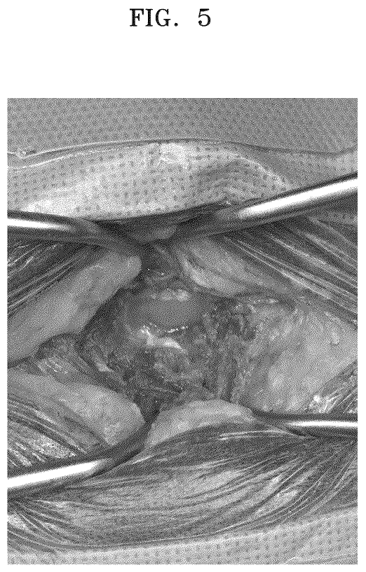

Next, as shown in FIG. 5, hemilaminectomy was performed on each of the four canine disc models, and 200 μl of the hydrogel patch for disc treatment prepared in Example 1 was applied to the compressed spinal cord area. After application, the time taken for the neurological grade to reach 0 (recovery period, day) was measured. Results are as shown in Table 2 below. As shown in Table 2, the neurological grade of all four cases of canine disc models became 0 within 14 days, confirming that application of the hydrogel patch was effective in treating discs.

TABLE 2

| | | Surgical site | | | |
|---|---|---|---|---|---|
| | | (Degree of | Neurological | | Recovery |
| Cases | Breed | compression) | grade (NG) | Surgery | period (day) |
| 1 | Dachshund | T12-13 Type I | 5 | Hemilaminectomy | NG5 → NG1 |
| | | (Severe) | | 410 μg/kg SGD101 | 5 days |
| | | | | | NG1 → NG0 |
| | | | | | 9 Days |
| 2 | French | L3-4, L4-5 Type I | 4-5 | Hemilaminectomy | NG4~5 → NG0 |
| | Bulldog | (Severe) | | 410 μg/kg SGD101 | 7 Days |
| | | L1-2, L2-3 Type II | | | |
| | | (Moderate) | | | |
| 3 | Dachshund | T13-L1 Type I | 4 | Hemilaminectomy | NG4 → NG2 |
| | | (Severe) | | 410 μg/kg SGD101 | 7 Days |
| | | | | | NG2 → NG0 |
| | | | | | 7 Days |
| 4 | Dachshund | L1-2 Type I | 4 | Hemilaminectomy | NG4 → NG0 |
| | | (Severe) | | 410 μg/kg SGD101 | 14 Days |

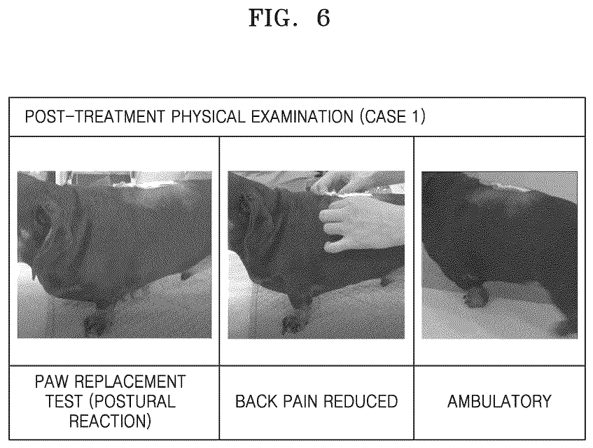

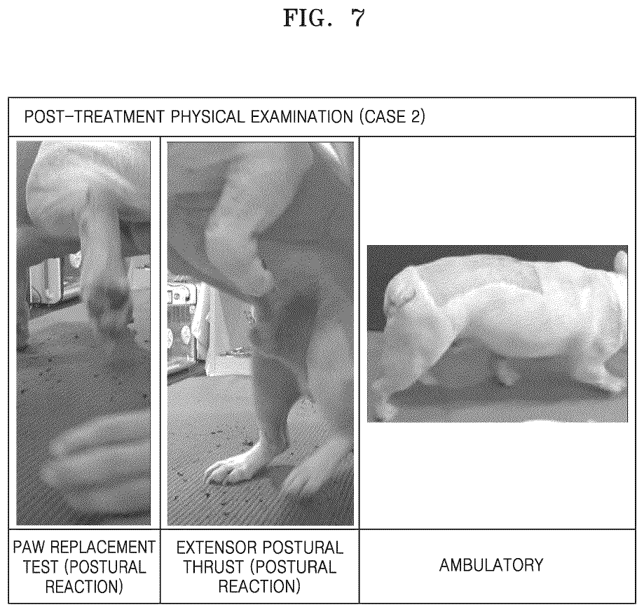

As a result of performing the check-up of the Dachshund disc model in Case 1 for 14 days after applying the hydrogel patch for disc treatment prepared in Example 1, as shown in FIG. 6, i) in the paw replacement test, both feet showed a postural response, ii) tactile pain in the lower back was relieved, and iii) in the ambulatory test, walking was possible on both feet. In addition, as a result of performing the check up of the French bulldog disc model in Case 2 for 14 days after applying the hydrogel patch for disc treatment prepared in Example 1, as shown in FIG. 7, i) in the paw replacement test, the left foot showed postural response, ii) in the extensor postural thrust test, the hind foot was straightened until the moment the foot touched the ground as the foot went down, and then, the natural movement of the hind foot to go backwards was shown after simultaneously touching the ground, and iii) in the ambulatory test, walking on the left foot was possible.

Additionally, 14 days after applying the hydrogel patch for disc treatment prepared in Example 1, the hind foot compression sensing ability was measured in four cases of canine disc models as follows. In detail, pressure was slowly applied to the hind foot of the canine disc model with forceps with a load cell attached thereto, and the pressure was measured using the load cell's computer program to apply pressure of up to 5 kg. When the canine disc model showed signs of pain while applying pressure of up to 5 kg, the pressing was stopped, and the pressure value at the time of discontinuation was measured to evaluate the compression sensing ability of the canine disc model. Pressure values were measured using forceps having a handle on which a load cell (iLoad Mini, Loadstar Sensors) was attached ([Gorney A. M. et al., 2016, J Vet Intern Med]). As shown in Table 3 below, the results showed that the compression sensing ability of the canine disc model was significantly restored by applying the hydrogel patch.

TABLE 3

| | Case 1 | | Case 2 | | Case 3 | | Case 4 | |
|---|---|---|---|---|---|---|---|---|
| | Before | After | Before | After | Before | After | Before | After |
| | treatment | treatment | treatment | treatment | treatment | treatment | treatment | treatment |
| Maximum | 5.0 | 0.8 | 1.9 | 1.0 | 2.2 | 0.9 | 1.6 | 0.8 |
| force (kg) | | | | | | | | |

Through these experiments, it was found that the hydrogel patch prepared in Example 1 was effective in treating disc disease and pain caused therefrom through recovery of paralyzed motor and sensory abilities in a disc disease animal model.

1. A pharmaceutical composition for preventing or treating herniated intervertebral disc or pain resulting therefrom, comprising as an active ingredient:
fibrin, or fibrin and fibrinogen;
laminin, or a laminin-derived peptide or protein; and
a hyaluronic acid or a salt thereof.

2. The pharmaceutical composition of claim 1, wherein the pharmaceutical composition is hydrogel or a hydrogel patch.

3. The pharmaceutical composition of claim 1, wherein the fibrin or the fibrinogen is included at a concentration of 0.1 mg/ml to 50 mg/ml, the laminin at a concentration of 1 μg/ml to 100 μg/ml, or the hyaluronic acid at a concentration of 10 μg/ml to 10 mg/ml.

4. The pharmaceutical composition of claim 1, further comprising thrombin.

5. The pharmaceutical composition of claim 1, further comprising a cell growth factor.

6. The pharmaceutical composition of claim 5, wherein the cell growth factor includes a nerve cell growth factor, a vascular endothelial cell growth factor, a fibroblast growth factor, a bone morphogenetic protein, an epidermal growth factor, a hepatocyte growth factor, a transforming growth factor, or a combination thereof.

7. The pharmaceutical composition of claim 2, wherein the hydrogel or the hydrogel patch does not include cells.

8. The pharmaceutical composition of claim 2, wherein the hydrogel or the hydrogel patch has a porous surface.

9. The pharmaceutical composition of claim 2, wherein the hydrogel or the hydrogel patch undergoes a reversible phase transition into a solid state, a semi-solid state, or a liquid state, depending on temperature.

10. The pharmaceutical composition of claim 1, wherein the herniated intervertebral disc is cervical herniated intervertebral disc or lumbar herniated intervertebral disc.

11. The pharmaceutical composition of claim 1, wherein pain caused by the herniated intervertebral disc is at least one selected from the group consisting of neck pain, lower back pain, coccydynia, and radiating pain.

12. A pharmaceutical composition for preventing or treating herniated intervertebral disc or pain resulting therefrom, comprising, as an active ingredient, hydrogel or a hydrogel patch wherein
the hydrogel or the hydrogel patch comprises:
fibrin, or fibrin and fibrinogen;
laminin, or a laminin-derived peptide or protein; and
a hyaluronic acid or a salt thereof.
